(19) United States
(12) Patent Application Publication
(10) Pub. No.: US 2020/0373011 A1
(43) Pub. Date: Nov. 26, 2020

(54) CARDIOMETRIC SPECTRAL IMAGING SYSTEM

(71) Applicant: DR. CECIL F. MOTLEY, ROLLING HILLS, CA (US)

(72) Inventor: DR. CECIL F. MOTLEY, ROLLING HILLS, CA (US)

(21) Appl. No.: 16/422,977

(22) Filed: May 25, 2019

(51) Int. Cl. G16H 50/20; G16H 50/70; G16H 40/20; G16H 40/67; A61B 5/024

(52) U.S. Cl. CPC G16H 50/20; G16H 50/70; A61B 5/02405; G16H 40/67; G16H 40/20

(57) ABSTRACT

This invention is based on the premise that every human or animal heart has a unique acoustic signature and that this signature has a statistical norm for each species (i.e. human, dog, cat, horse, pig, etc.). Further, a deviation from this statistical acoustic signature norm, is an indication of a cardiac abnormality or disease. This biophysical model gives rise to a diagnostic system by which a patient's heart sound signature can be compared to known abnormal heart sound signatures to provide early detection of cardiac morbidity on a non-intrusive basis. This invention is for the method, apparatus, and system used to implement this process.

The technique, henceforth referred to as Cardiometric Spectral Imaging, is the non-invasive method and system from which 3D contours are derived from time-frequency analysis of the heart sounds (S1 thru S4) signatures individually. The 3D contour data is used as input to a correlation process that yields a feature set that is used as input to a Deep Learning Neural Network. These processes are cognitively managed using Artificial Intelligence based pattern correlation searches and multiple-output supervised neural networks. Additionally, the system computes a cardiac severity rating which predicts the degree of advancement of the early diagnosed heart condition.

This invention is implemented using a special acoustic sensor, a sensor interface module, an associated SmartPhone or personal computer, a centralized server farm that executes the Artificial Intelligence and Deep Learning Neural Network algorithms, an updateable cardiac sounds contour template database, and advanced signal processing technology. The system operation involves placing a special acoustic sensor on the subject's chest near the apex of the heart. The signals from the sensor are digitized and pre-processed by a sensor interface module. The interface module then connects to a SmartPhone or Personal Computer where the data is packaged and sent to the Cardiometric Processing Center Server Farm where the time-frequency analysis, Deep Learning Neural Network algorithms, Wavelet Transforms, and pattern correlation processes are executed. The diagnostic results and cardiac state are sent back to the SmartPhone or Personal Computer for review by healthcare personnel. This invention emulates the auscultation performed by healthcare professionals using a stethoscope without the need for them to have perfect hearing at very low frequencies and expert recognition skills for identifying abnormal heart sound patterns. The invention is useable in a remote, home or clinical environment.

Additional problems solved by this invention include normalization of heart sound signature data for young children, women, older persons and DNA imposed heart signature parameters (i.e. tonal ranges, heart rates, gap signals between S1 thru S4 heart sounds, lung noise, and external noise or vibration). The invention includes a comprehensive data set of normal and abnormal heart sound signatures associated with all genders and ages. New and unknown heart sound signatures encountered by the Cardiometric Spectral Imaging system are automatically included in the template database and are labeled once an independent diagnosis has been established.

### PUBLICATION CLASSIFICATION

| | |
|---|---|
| A61B 5/00 | 2006.01 |
| A61B 5/02 | 2006.01 |
| A61B 7/00 | 2006.02 |
| A61B 7/02 | 2006.01 |
| A61B 7/04 | 2006.01 |
| A61B 5/0205 | 2013.01 |
| A61B 5/01 | 2013.01 |

### REFERENCES CITED

| | | |
|---|---|---|
| U.S. Pat. No. 6,418,346 B1 | Jul. 9, 2002 | Nelsen et al. |
| U.S. Pat. No. 6,650,940 B1 | Nov. 18, 2003 | Zhu et al. |
| U.S. Pat. No. 7,052,466 B2 | May 30, 2006 | Scheiner et al. |
| U.S. Pat. No. 6,599,250 B2 | Jul. 29, 2003 | Webb et al. |
| U.S. Pat. No. 9,610,059 B2 | Apr. 4, 2017 | Christensen et al. |
| U.S. Pat. No. 6,953,436 B2 | Oct. 11, 2005 | Watrous et al. |
| U.S. Pat. No. 8,641,632 B2 | Feb. 4, 2014 | Quintin et al. |
| U.S. Pat. No. 7,508,307 B2 | Mar. 24, 2009 | Albert |
| U.S. Pat. No. 7,983,744 B2 | Jul. 19, 2011 | Ricci et al. |
| U.S. Pat. No. 8,690,789 B2 | Apr. 8, 2014 | Watrous |
| U.S. Pat. No. 8,790,264 B2 | Jul. 29, 2014 | Sandler et al. |
| U.S. Pat. No. 9,161,705 B2 | Oct. 20, 2015 | Tamil et al. |
| U.S. Pat. No. 9,060,683 B2 | Jun. 23, 2015 | Tran |
| U.S. Pat. No. 9,168,018 B2 | Oct. 27, 2015 | Pretorius et al. |
| U.S. Pat. No. 10,136,861 B2 | Nov. 27, 2018 | Ong et al. |
| US 2004/0096069 A1 | May 20, 2004 | Chien |
| US 2008/0232605 A1 | Sep. 25, 2008 | Bagha |
| US 2008/0228095 A1 | Sep. 18, 2008 | Richardson |
| US 2008/0146276 A1 | Jun. 19, 2008 | Lee |
| US 2011/0190665 A1 | Aug. 4, 2011 | Bedingham et al. |
| US 2004/0138572 A1 | Jul. 15, 2004 | Thiagarajan |
| US 2013/0289378 A1 | Oct. 31, 2013 | Son et al. |
| US 2004/0260188 A1 | 21/23/2004 | Syed et al. |
| US 2006/0161064 A1 | Jul. 20, 2006 | Watrous et al. |
| US 2008/0103403 A1 | May 1, 2008 | Cohen |
| US 2008/0013747 A1 | Jan. 17, 2008 | Tran |
| US 2010/0094152 A1 | Apr. 15, 2010 | Semmlow |
| US 2018/0214030 A1 | Aug. 2, 2018 | Migeotte et al. |
| US 2018/0333094 A1 | Nov. 22, 2018 | Palaniswami et al |

### BACKGROUND OF THE INVENTION

### 1. Field of the Invention

This invention meets the need to eliminate early cardiac abnormality detection limitations due to a Physician or Health Care Professional's inability to auscultate hearts sounds in the 3 Hz to 40 Hz frequency range using an acoustic or electronic stethoscope. This frequency range is where most early irregular heart sounds and murmurs occur. The method, apparatus, and system described in this disclosure minimizes auscultation errors by applying three dimensional (3D) time-frequency analysis of the S1 thru S4 heart sound components separately and comparing the results using pattern recognition techniques to a library of known heart sound abnormality and disease 3D contours. This process performs a non-invasive computer assisted real-time diagnostic procedure henceforth referred to as Cardiometric Spectral Imaging (CSI). Deep Learning Neural Network (DLNN) based Artificial Intelligence (AI) algorithms are used to identify the cardiac abnormality with a 97% accuracy.

### 2. Background Art

The concept of using heart sound data, known as Phonocardiograms (PCG), as a medical diagnostic tool originated in 1924 which was essentially an electronically amplified stethoscope. The resulting sounds and graphical representations were so modified that physicians had to alter their listening techniques which distracted from the usefulness of the device. Various versions of the electronic stethoscope were created up to the 1980s when the centers for Medicare and Medicaid stopped paying for PCGs because they were outmoded and had very little diagnostic benefit. All insurance companies also stopped paying for the test. As a result, virtually all physicians stopped using PCGs as a part of diagnostic workups. To date PCGs have not provided the diagnostic effectiveness required to provide an early accurate diagnosis based on auscultation alone. Recent versions of automated stethoscopes include techniques to abstract cardiac features and reduce noise from the heart sound signal. Additionally, the application of neural network technology to access diagnostic data from the PCG data is included in recent patents.

In disclosure US 2008/0232605 A1 an electronic stethoscope is described that utilizes improved amplification, noise suppression, signal processing techniques and wireless communication via Bluetooth links. Although improvements over early electronic stethoscopes are included, it still has the deficiencies of not enabling the user to hear separate S1 to S4 sounds in the 3 Hz to 40 Hz frequency range and automatically diagnosing early associated heart abnormality.

Current electronic stethoscope technology (US 2008/0232605 A1, US 2008/0228095, US 2008/0013747 A1, US 2004/40260188) includes amplified devices, state-of-the-art sound sensors, ambient noise reduction technology, frictional noise reduction, listening range frequency selection, time-frequency analysis, feature extraction, neural networks to detect patterns in the PCG signal, and up to 24× sound amplification. Other current approaches to using PCG data management include SmartPhone applications (US 2008/0146276 A1) that use microphones and associated processing to store heart sound data for later playback. Although improvements over early electronic stethoscopes are included, they still have the deficiencies of the early electronic units (i.e. lack of ability to provide early diagnosis of a full range of heart abnormalities and disease).

In disclosure US 2004/0138572 A1, a method and apparatus is described that uses S1 and S2 heart sounds to determine an energy envelope for each of a plurality of regions in the signal for the purpose of determining the characteristics of several areas of the heart. This method does not provide the diagnostic accuracy required by current diagnostic systems.

Several implantable devices have been disclosed, (U.S. Pat. No. 6,650,940 B1, U.S. Pat. No. 7,052,466 B2, U.S. Pat. No. 6,599,250 B2, US 2013/0289378 A1) that provide rigorous detection and analysis of heart sounds, however, surgical procedures are required to place the sensors in the heart.

In disclosure U.S. Pat. No. 9,610,059 B2, a system and device is described that uses a non-invasive acoustical technique to collect heart sound data and locally process the data to assist in diagnosis. This method also does not provide the diagnostic accuracy for a comprehensive collection of cardiac conditions required by current diagnostic work ups.

In disclosure U.S. Pat. No. 10,136,861 B2, a system and method is described that uses a plurality of patient health data to train a neural network for the purpose of predicting patient survivability associated with an acute cardiopulmonary event. This method uses data collected by other means and equipment as input to its neural network and therefore not comprehensive with respect to diagnosis of a full range of heart abnormalities or diseases.

In disclosure US 2018/0333094 A1, a system and device is described for use by stroke victims to aide in the identification of neuro-cardiological disturbances. This is basically a portable monitoring device for detecting and recording a specific heart activity associated with strokes. This method does not provide a full range of heart abnormality or disease early diagnostics using non-invasive acoustic techniques.

In disclosure US 2018/0214030 A1, a portable device is described that provides multi-dimension kineticardiography data during exercise or other activity. This method does not provide a full range of heart abnormality or disease early diagnostics using non-invasive acoustic techniques.

In disclosure U.S. Pat. No. 9,168,018 B2, a method is described for extracting and classifying feature data of a normal and abnormal heart sound signal. Neural network technology is used to diagnose a selected heart pathology (murmurs only). This method does not provide a full range of heart abnormality or disease early diagnostics for a simultaneous plurality of heart sound sensors using non-invasive acoustic techniques. The neural net does not have the ability to learn from large quantities of data from the plurality of sensors. Additionally, the preprocessing of S1 thru S4 heart sounds and their associated gaps are not included in the process which is necessary for early diagnosis.

In disclosure U.S. Pat. No. 9,060,683 B2, a portable wrist device that transfers patient health data to authorized users is described. This device does not provide a full range of heart abnormality or disease early diagnostics using non-invasive acoustic techniques.

In disclosure U.S. pat. No. 9,161,705 B2, a portable device is described that uses ECG data transferred to a clinical environment using a cell phone to detect abnormal heart conditions, especially ischemia in advance of a heart attack. This method does not provide a full range of heart abnormality or disease early diagnostics using non-invasive acoustic techniques.

In disclosure U.S. pat. No. 8,790,264 B2, a method is described that uses the difference between the first and second frequency of heart sound data to determine the condition of the heart. This method does not provide a full range of heart abnormality or disease diagnostics using non-invasive acoustic techniques.

In disclosure U.S. pat. No. 8,690,789 B2, a system and method is described that automatically maps time-frequency analyzed PCG data to systolic and diastolic abnormalities associated with murmurs. This method does not provide a full range of heart abnormality or disease early diagnostics using non-invasive acoustic techniques.

In disclosure US 2010/0094152 A1, a system and method is described that uses spectral analysis of the S1 and S2 heart sounds to create a vector set for comparison to vector sets of known heart states. S3 and S4 are not detected or include in the diagnostic process and therefore this system does not provide a full range of heart abnormality or disease detection for early diagnostics using non-invasive acoustic techniques.

In disclosure U.S. Pat. No. 7,983,744 B2, a neural network structure is described that is optimized for operating an implantable cardiac device. This method and device does not provide a full range of heart abnormality or disease early diagnostics using non-invasive acoustic techniques.

In disclosure U.S. Pat. No. 7,508,307 B2, a patient health monitoring device is described that senses the need for a medical response. This method does not provide a full range of heart abnormality or disease early diagnostics using non-invasive acoustic techniques.

In disclosure US 2008/0103403 A1, a method and system is described that uses ECG signal data analysis and neural nets to classify patient heart states. This technique does not use acoustic sensors and deep learning neural nets and therefore unable to diagnose abnormal or disease heart conditions with greater than 97% accuracy.

In disclosure U.S. Pat. No. 8,641,632 B2, a method is described to monitor the efficacy of anesthesia using cardiac baroreceptor reflex function. This method does not provide a full range of heart abnormality or disease early diagnostics using non-invasive acoustic techniques.

In disclosure US 2006/0161064 A1, a method is described for diagnosis of heart murmurs using energy calculations of spectral components of heart sound signal. This method does not provide a full range of heart abnormality or disease early diagnostics using non-invasive acoustic techniques.

In disclosure U.S. Pat. No. 6,953,436 B2, a method is described for extracting and evaluating features from cardiac acoustic signals. This method uses Wavelet analysis and a neural net to determine the status of heart murmurs. Although this method improves early murmur detection and classification, it still has the deficiencies of not including separate S1 to S4 sounds analysis plus their gaps in the 3 Hz to 40 Hz frequency range and correctly diagnosing early associated heart abnormalities and disease. Additionally, the method does not provide a full range of heart abnormality or disease early diagnostics using non-invasive acoustic techniques.

To date there is still a need for a non-invasive heart monitoring and diagnostic system that is not limited by Physicians and Health Care Professionals ability to auscultate S1 thru S4 heart sounds in the 3 Hz to 40 Hz frequency range where early abnormal heart sounds are most commonly present. The technique used in this invention compares the time-frequency representations of a patients four heart sounds (S1 thru S4) to a library of known normal, abnormal and diseased 3D time-frequency contours. Once a high probability match is determined, the associated diagnosis and heart state rating is reported. The primary challenge associated with this technique is the unique variation in the patient sound groups, heart repetition rate, and tonal differences between male, female, and age of the patient. This invention uses AI Deep Learning Neural Networks to emulate the Health Care Professional's ability to use their cognitive capability to make an identification of the sound signature presented by the four heart sounds that lead to an accurate diagnosis regardless of the patient type or age. The disclosed CSI method, system, and apparatus invention fulfills this need by applying state-of-art acoustic vibration sensors, multi-resolution wavelet based digital signal processing, multi-layer (10 to 100 layers) DLNN, 3D correlation pattern recognition, mobile devices and robust remote server farm supercomputers to elevate electronic auscultation diagnosis to a level consistent with modern diagnostic techniques.

### SUMMARY OF THE INVENTION

This invention achieves the above mentioned objective by eliminating the need for Physicians or healthcare professionals to hear separate S1 to S4 heart sound components in the 3 Hz to 40 Hz frequency range and correctly associate them to specific cardiac abnormalities and diseases. The CSI System uses motion vibration sensors designed for the building earthquake sensing industry to detect the heart sounds, thus providing the ability to sense very low frequency sounds at extremely low amplitude levels. Signals from these sensors are digitized and packaged for transfer to a remote Cardiometric Processing Center (CPC) where each heart sound component is windowed, time-frequency analyzed using a Continuous Wavelet Transform (CWT) and displayed as a 3D contour (FIG. 3). The Cardiometric Processing Center has a plurality of contour references (template database) that represent known cardiac abnormalities and diseases. The subject data is then compared to these standardized contour templates. AI directed correlation mathematics, and DLNN are used to manage the contour analysis and update the template library database. When there is a high probability that a match has been detected, the corresponding diagnostic information and associated cardiac state rating (CSR) is sent back to the remote user. Additionally, the subject's data package is stored at the processing center for future use and historical comparison. DLNN techniques continuously update and optimize the standardized contour templates thus including any new heart abnormality encountered.

The user's apparatus that captures the heart sound data consists of the specialized vibration sensor and a sensor interface module (SIM) attached to the USB port of an associated SmartPhone or Personal Computer that is executing the pre-processing software. This remote apparatus connects to the Cardiometric Processing Center via the internet cloud, WAN or LAN using encrypted communication, thus abiding by HIPPA regulations. The processing center consists of a plurality of high performance computers and storage devices. Supercomputers are used to execute the AI and DLNN algorithms and associated correlation and diagnosis identification process.

### BRIEF DESCRIPTION OF THE DRAWINGS

The invention is illustrated by way of example and not by way of limitation in the figures of the accompanying drawings in which like references indicate similar elements. It should be noted that references to “an” or “one” embodiment in this disclosure are not necessarily to the same embodiment and such references mean at least one.

FIG. 1 illustrates the range of heart sounds audible to the normal human ear.

FIG. 2 illustrates the S1 thru S4 sounds produced by the human heart.

FIG. 3 illustrates an example of a 3D spectral contour of the S2 heart sound.

FIG. 4 illustrates the Cardiometric Spectral Imaging System Block Diagram.

FIG. 5 illustrates one embodiment of the special acoustic low frequency sensor.

FIG. 6 illustrates the Block Diagram of one embodiment of the Sensor Interface Module.

FIG. 7 Cardiometric Spectral Imaging Diagnosis and Learning Process.

FIG. 8 AI Correlation and Neural Net.

FIG. 9 CSI Neural Net Activity Function.

FIG. 10 illustrates the CWT computations and heart sound data pre-processing.

FIG. 11 CWT Morlet Wavelet

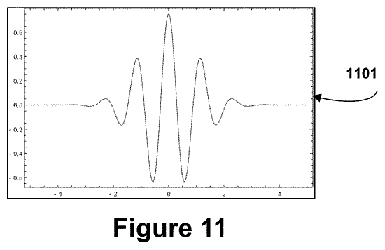

FIG. 12 illustrates the CSI Central Processing Center Server Farm Functional Elements.

FIG. 13 illustrates the CSI Neural Net learning process.

### BRIEF DESCRIPTION OF THE EQUATIONS

Mathematical calculations associated with this invention are explained with the aid of the following equations:

1) Equation 1 Algebraic and Matrix representation of the Deep Learning Neural Net Nodes.

2) Equation 2 Continuous Wavelet Transform

3) Equation 3 Correlation Calculation

4) Equation 4 Cardiac Severity Calculation

5) Equation 5 Neural Net output function

6) Equation 6 Least Squares loss function

### DETAILED DESCRIPTION

The beating heart of a human or animal provides an excellent acoustic source, the properties of which are determined by its physical structure, the body structure of its host, and its beating rate. Essentially, the heart and host configuration is synonymous with an audio speaker enclosed in an associated speaker cabinet. Hence, the audio emitted from the unit is spectrally shaped by its physical and surrounding structure. Sonar systems employed by the NAVY have used acoustic analysis to identify the presence and characteristics of underwater targets for many decades and their algorithms are well understood. This invention is an embellished adaptation of this technology for the purpose of identifying the properties of the beating heart acoustic signature. This signature is used to test the current heart state as well as detect early signs of cardiac abnormality or disease.

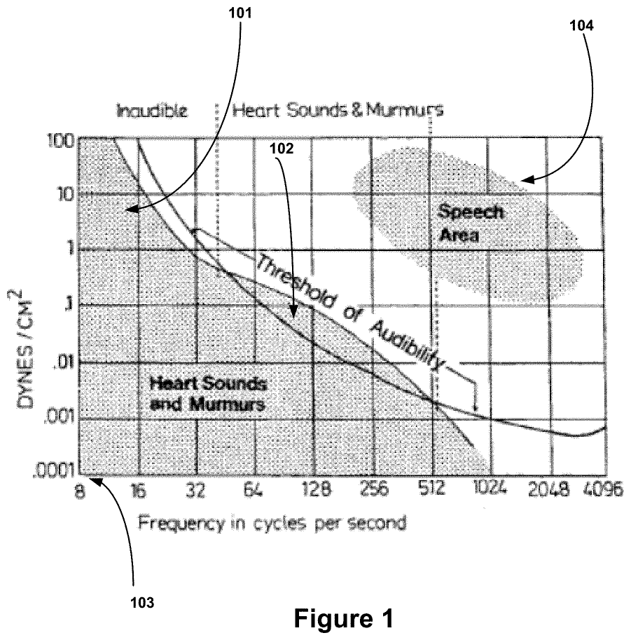

With reference to FIG. 1, the human heart produces sounds and murmurs in the 3 Hz 101 to 1000 Hz 102 range. Auscultation of these sounds is limited by a Physician's or healthcare professional's ability to hear sounds between 3 Hz and 40 Hz with a stethoscope where abnormal sounds and murmurs are easily identified. The auditability range for humans is in the 30 Hz to 15 KHz range 102. Therefore detectability of many heart abnormalities that are manifested in sounds below 30 Hz are not detected using stethoscope auscultation.

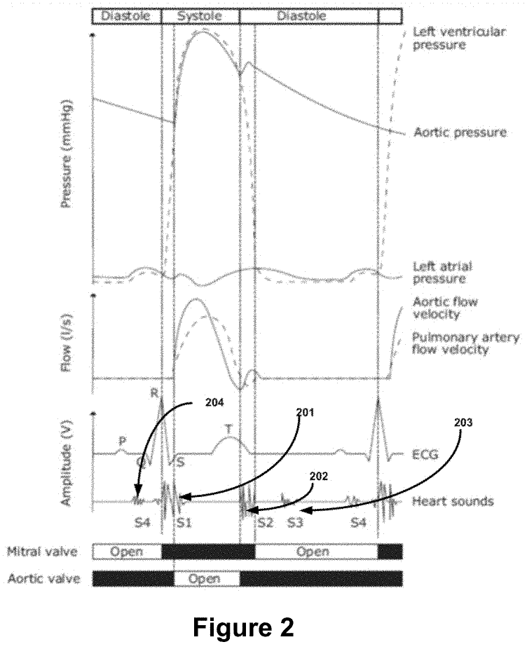

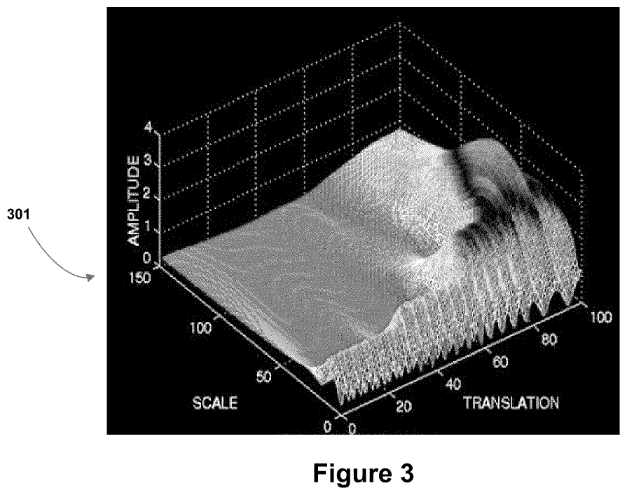

This invention eliminates this problem by applying the CSI system described in this disclosure. Heart abnormalities and disease cause the heart to have specific sound time-frequency patterns that deviate from the statistical norm. Instead of relying on humans to hear and cognitively sort out these patterns, a special vibration sensor and associated computers executing wavelet time-frequency analysis, digital signal processing and AI algorithms correctly detect heart sound components as low as 3 Hz using computer implementation of three dimensional (3D) time-frequency analysis of S1 thru S4 heart sounds 201-204 (FIG. 2). Each heart sound component has a unique spectral contour for each human individual. The combination of the S1 thru S4 heart sound time-frequency contours is a unique signature for each individual. Although each individual has a unique set of CSI contours, there are common heart sound signatures that indicate when an abnormal or diseased condition is present. This invention exploits the deviation from these common norms in the CSI 3D contours 301 (FIG. 3) to identify the presence of an abnormal or diseased heart state. This detection process is executed using deep learning semantic networks manifested as goal trees and neural nets. An additional benefit of this type analysis is the ability to rate the severity of the heart state on a cardiac severity rating scale, “CSR”, which is easily interpreted for clinical applications.

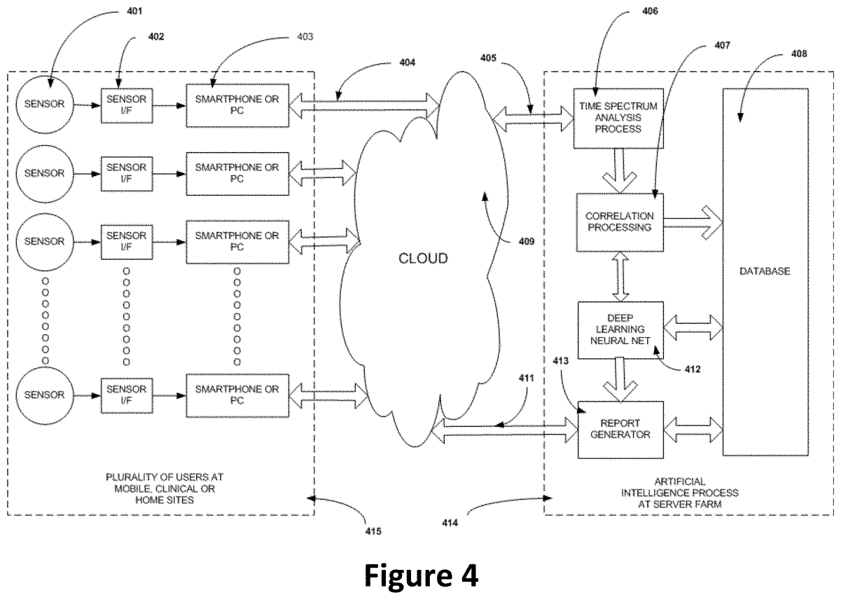

With reference to FIG. 4, the invention includes a plurality of remote portable heart sound monitoring apparatus 415 that consists of special acoustic sensors, 401 a sensor interface module (SIM) 402 and associated SmartPhone or PC 403. A Central Processing Server farm 414 at a remote location is linked to the SmartPhone or PC through a local or wide area network (public or private) 404 to provide the signal processing functions 406, 407, and 412 required to create the 3D contours. Additionally, the Central Processing Center is the location of the reference diagnostic contours database 408 as well as patient Cardiometric data. The entire process is managed by an AI manager.

One embodiment of this invention uses the public internet cloud 409 for the link between the plurality of remote SmartPhones and the Cardiometric Processing Center. A second embodiment of this invention uses a local area network (LAN) of a hospital, diagnostic center, or medical care facility to link the plurality of remote Smartphones to the Cardiometric Processing Center 414. A third embodiment of this invention uses a private network (WAN) to link the SmartPhones to the Cardiometric Processing Center 414 server farm. For each embodiment the results 411 from the Cardiometric Processing Center are sent back to the remote SmartPhone or PC through the Cloud, LAN, or WAN. Optionally, reports are e-mailed from the server farm 414 directly to the patients Physician or associated health care professional. A forth embodiment of this invention uses only localized resources to collect the heart sound signal, perform the CSI 3D analysis, perform pattern recognition, and AI based computer assisted diagnosis. In this structure, the heart sound collection and high performance server processing equipment are co-located. This forth embodiment is consistent with a centralized clinical imaging facility where the procedure is performed by radiologist or healthcare professionals.

1. Special Acoustic Sensor

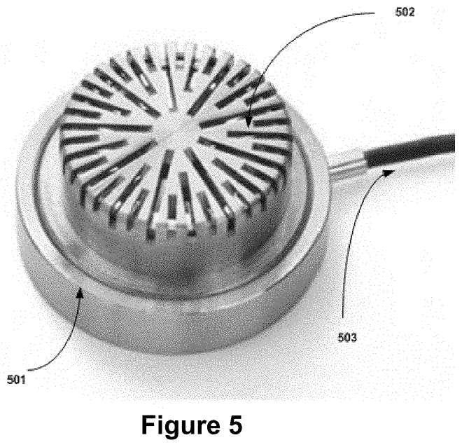

The CSI system special acoustic sensor is a highly sensitive vibration sensor with a flat frequency response in the 3 Hz to 1000 Hz range. One embodiment of this invention uses a G.R.A.S. 47AD unit (FIG. 5) with the following general specifications:
Frequency Range: 3.15 Hz to 10 Khz
Dynamic Range: 18 dB(A) to 138 dB
Preamplifier: Built-in CCP
Sensitivity: 50 mV/Pa

The microphone 501 is a pre-polarized unit with a built in low noise constant current powered preamplifier 502 and an automatic transducer identification circuit 503.

2. Sensor Interface Module

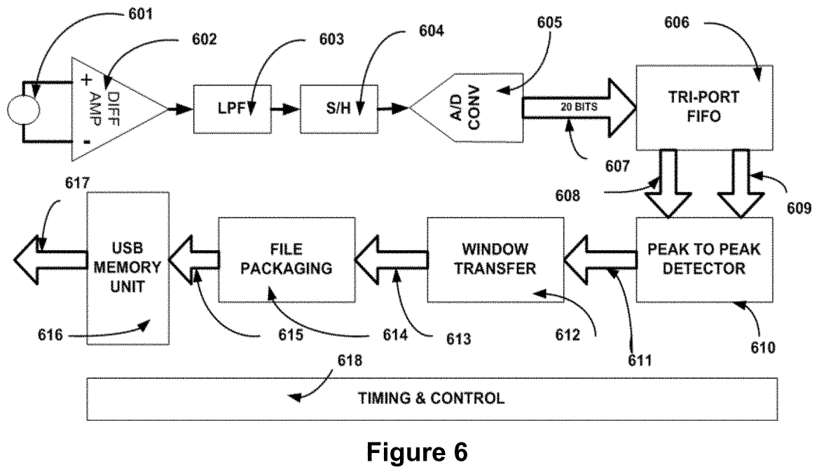

The CSI remote unit Sensor Interface Module block diagram is shown in FIG. 6. This module provides the pre-processing of the heart sound signals for transport to the Cardiometric Processing Center by the SmartPhone or PC. One embodiment of the SIM uses a very low frequency low noise differential amplifier 602 to increase the heart sound signal from the acoustic sensor 601 to at least 5 volts peak-to-peak. The signal is then filtered with a 1 KHz low pass filter 603 to prevent aliasing by the sample and hold 604 and the high resolution analog to digital conversion process 605. The process of separating the individual S1 through S4 sound component is performed using a Quad-Port First in First out (FIFO) 606 buffer. The FIFO provides the facility to detect and separate the signal windows (signal+gap) associated with each of the S1 through S4 sound components. The peak detector array 610 locates the S1 through S4 repeat pattern and separates each component into a separate window 612 which is packed into separate files 614 and transferred to a memory array 616 that emulates a USB memory device compatible 617 with the SmartPhone or PC.

3. Deep Learning Artificial Intelligence

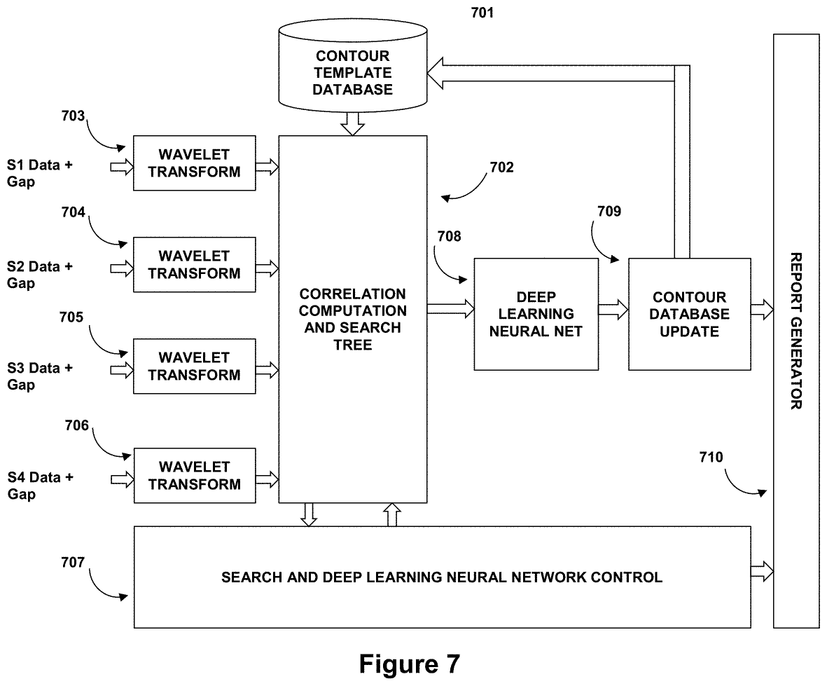

This invention uses Deep Learning Neural Network (DLNN) Artificial Intelligence (FIG. 7) to manage the Wavelet Time-Frequency Analysis parameters 703, 704, 705, and 706, the 3D Contour correlation process 702, the DLNN Tree 708 and the 3D Contour database updates 701, 707, and 709. One embodiment of the invention involves the algorithms that process the individual S1 thru S4 data and emulate an improvement in the cognitive acoustic analysis normally done by healthcare personnel using a stethoscope. The goal of the AI algorithms is to determine which known heart diagnosis best fits the target patient heart state. This invention contains 23 heart sound signatures that correspond to known heart abnormalities or disease.

The DLNN based diagnostics is a three stage process. The first stage of the AI process involves converting the acoustic heart sound signal into an image map that is manageable by DLNN algorithms 707. One embodiment of this invention uses a Continuous Wavelet Transform Equation (2) to analyze the acoustic heart signal in time for its frequency content and create a 3D time-frequency image of the signal. This approach provides a multiresolution analysis (MRA) where the signal is analyzed at different frequencies with different resolutions. The MRA is designed to give good time resolution and poor frequency resolution at higher frequencies and good frequency resolution and poor time resolution at low frequencies where abnormalities are most detectable. This approach is consistent with heart sound signals. By way of example, an isolated S2 cardiac time-frequency 3D mapping 301 is illustrated in FIG. 3.

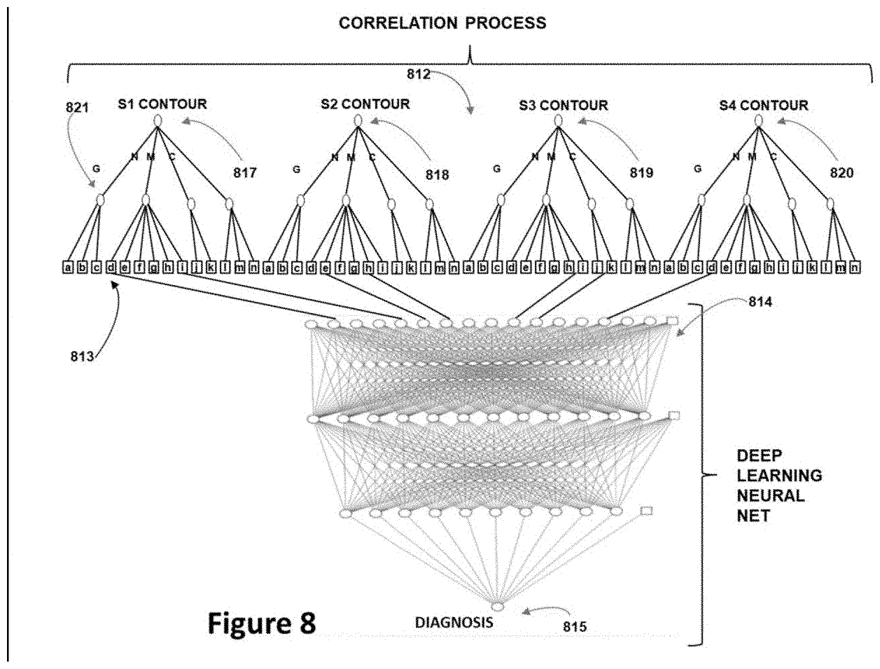

With reference to FIGS. 7 and 8, the S1 thru S4 signals including the information in the gaps between the signals provide the input data to the CWT 703, 704, 705, and 706. This invention separates the S1 thru S4 signals into four separate data sets with the goal of reducing the processing capacity required to correctly identify a particular early heart abnormality or disease. The CWT performs the MRA process on each S-signal individually. This is the 3D mapping process that is used in the correlation process 702 where the patient input data is compared to the known abnormality contours 701. These contours form the root nodes 708 of the identification process. Ideal models of heart sounds of known heart abnormalities or disease are processed using MRA and make up the image template data base 701 used in the DLNN training data and actual patient diagnostic process. The template categories are normal, murmur, clicks, and gallop which reflect types of heuristic sounds observed by health care professionals using a stethoscope and form the second node level 821 in the identification process. Training data from 500 human subjects with known heart abnormalities or disease are used to calibrate the system.

The second stage of the process involves computing the correlation values between patient cardiac sound data and the template database. The output of this process is a vector of numbers that reflect how well each of the patients S-sounds compares to the S-sounds of known abnormalities or disease. This vector of numbers provides the input nodes 808 to the DLNN.

The third stage of the process involves training, validating and testing the DLNN (FIG. 8). One embodiment of this invention uses a 10 layer DLNN with a 23 node output layer 814. The DLNN inputs data from the four correlation processes 812 (96 feature data set).

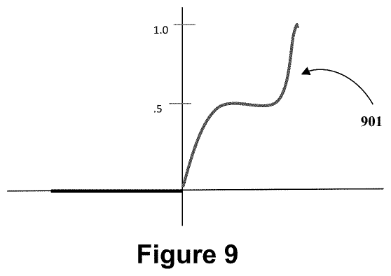

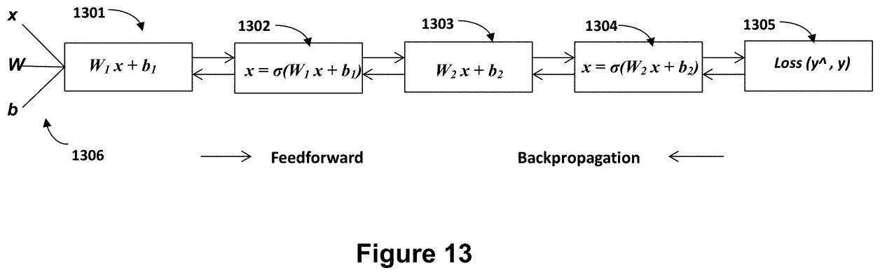

In procedural English, overall the process is as follows:
Step 1: Individually digitize S1 thru S2 heart sounds are mapped into digital image contours using a multiresolution CWT with a “Morlet” mother wavelet 1101.
Step 2: The CWT contours are normalized to a standard form by dilation or compression which involves adjusting the CWT parameters (scale and translation).
Step 3: Execute training process (FIGS. 13) 1301, 1302, 1303, 1304, and 1305 using heart sound signature training data contour sets 812 into a (10 to 100) layer Deep Learning Neural Network 814 to select the weights and biases that provide minimum loss (i.e. closeness of match between the data set and diagnostic classification). Steepest Gradient and Backpropagation are used to fine tune the weights and biases.
Step 4: Perform correlation computations 1002 between the patient heart sound signatures and the reference contour templates.
   Group Correlation computations into four categories.
      Normal Signatures—N
      Murmur Signatures—M
      Click Signatures—C
      Gallop Signatures—G
   Each correlation computation produces a number between 0 and 1.
   Form reference template correlation value vectors reference database 1001.
   Input the correlation numbers into the Deep Learning Neural Net Tree 1005.
   Node functions are given by Equation (1) and the activity function is described in (FIG. 9), 901 and henceforth referred to as Worm_1. This function is specifically designed to optimize the threshold process between the neural net hidden layer nodes.
Step 5: Use the DLNN tree to determine which diagnosis best fits the patient's heart sound signature pattern with at least 97% certainty.
Step 6: Compute the CSR, a value between 0 and 1, based on correlation data and resulting diagnosis Equation (4). Early onset of abnormal conditions is manifested by progressively higher CSR numbers.
Step 7: Update the contour database 709 for the cases where no high degree of diagnostic certainty can be determined for the patient input data set. The new condition entry is later titled after validation by other diagnostic procedures.
Step 8: Create a report which contains all related diagnostic data 710.
   One embodiment of this invention is capable of identifying the following heart sound patterns, thus diagnosing the associated normal or abnormal condition with a 97% accuracy:

| | |
|---|---|
| Single S1 S2 | Normal |
| Split S1 | Normal |
| Mid Systolic Click | Mitral Valve Prolapse |
| Early Systolic Murmur | Acute Mitral Regurgitation |
| Mid Systolic Murmur | Mitral Regurgitation (Coronary Artery Disease) |
| Late Systolic Murmur | Mitral Regurgitation (Mitral Valve Prolapse) |
| Holosystolic Murmur | Mitral Valve Prolapse with Regurgitation |
| S4 Gallop | Left Ventricular Hypertrophy |
| S3 Gallop | Both Normal and Cardiomyopathy |
| Systolic Click + Late Murmur | Mitral Valve Prolapse with Mitral Regurgitation |
| S4 and Mid Systolic Murmur | Ischemic Cardiomyopathy + Mitral Regurgitation |
| S3 and Holosystolic Murmur | Dilated Cardiomyopathy with Regurgitation |
| Mitral Snap and Diastolic Murmur | Mitral Stenosis |
| Normal S1 S2 | Supine Normal |
| Systolic Murmur with Absent S2 | Severe Aortic Stenosis |
| Early Diastolic Murmur | Aortic Regurgitation |
| Systolic and Diastolic Murmurs | Combined Aortic Stenosis and Regurgitation |
| Single S2 | Normal in Elderly |
| Split S2 Persistent | Complete Right Bundle Branch Block |
| Split S2 Transient | Normal |
| Ejection Systolic Murmur | Innocent Murmur |
| Ejection Systolic Murmur Split S2 | Arterial Septal Defect |
| Ejection Systolic Murmur + Click | Pulmonary Valve Stenosis |

This invention is capable of identifying these patterns in men, women or children of any age by adjusting the scale dilation or compression of the CWT. The invention is also capable of identifying patterns not in the CSI contour database 701 and auto classifying them based on other diagnostic procedures.

4. CSI Preprocessing and Training Data Preparation

One embodiment of the invention uses synthetically produced Training Data of the heart sound patterns for the DLNN. This data henceforth referred to as Cbad_1 and Ctest_1 is created by digital emulation of the aforementioned heart sounds resulting from normal, abnormal, and diseased heart states. The Training Data is used to initialize the DLNN prior to imputing hundreds of diagnosed heart abnormality data sets. These data sets correspond to known diagnosed cardiac conditions and are primarily used to tune the weights and bias in the neural network tree. The data sets are divided into training data, validation data, and test data.

5. CWT and Correlation Computations

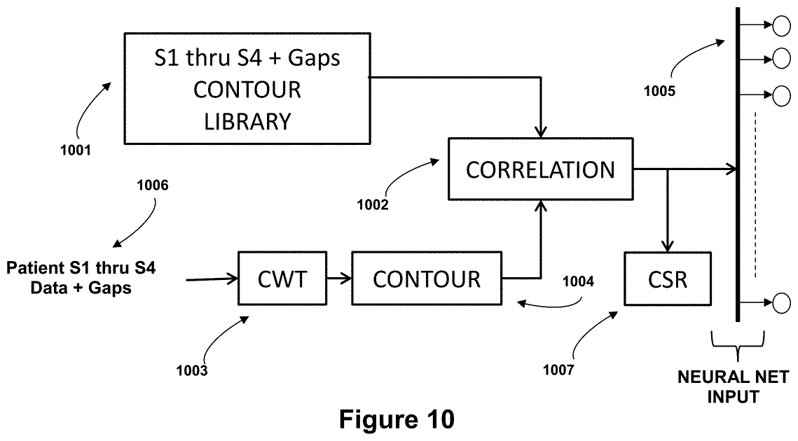

In reference to FIG. 10, one embodiment of this invention uses CWT to map the heart sounds S1 thru S4 plus gaps 1006 into individual 3D contours 1001 and 1004. A correlation process 1002 is used to prepare the DLNN 1005 input data. The correlation process compares the cardiac sound contour library templates with patient S1 thru S4 heart sounds data. This architecture greatly reduces the number of input data points 1005 required by the system without sacrificing identification accuracy. In this embodiment of the invention, ninety two (92) contour templates are compared to the patient contour data (i.e. four sets of twenty three (23) which corresponds to the 23 diagnosable cardiac abnormalities in the contour library).

The mathematical expression for the CWT is provided in Equation 2 where s is the scale, t is translation and the value of the CWT is the magnitude between 0 and 1. A “Morlet” Wavelet 1101 is used in the CWT due to its close relationship with human perception, both hearing and vision. A discretized version of the CWT calculations allows execution by a digital computer. The CWT calculations are used to form the contours used in the first stage of the identification process. The dimensions of the contours are Scale, Translation and Magnitude. These contours represent spectral properties of the heart sounds, which are unique for each beating heart. A library of synthetically implemented heart sound contours is used as templates for comparison with real patient data.

The mathematical expression for the correlation process is provided in Equation 3. Correlation between the patient data set and each of the 92 heart sound templates is computed. The resulting 92 correlation values 812 form the input nodes of the neural network 814 and are indicative of how well the patient heart sound contours match each of the 23 heart conditions. Once a high probability diagnosis match is determined by the neural network, the cardiac severity rating (CSR) is computed as the maximum correlation values from each of the sounds (S1 thru S4) averaged (Equation 4).

6. CSI Processing Center

The implementation of this invention is primarily possible due to the recent availability of super computers, Terabyte storage, and high bandwidth online connectivity. A diagnostic DLNN that has a 97% accuracy, requires processing neural trees with hundreds of nodes and branches. With the availability of 3.00 GHz twelve core twelve thread processors, 30 Megabyte on chip cache, and Terabyte high speed memory, it is possible to easily process neural trees with 10⁶ nodes and 10² hidden layers.

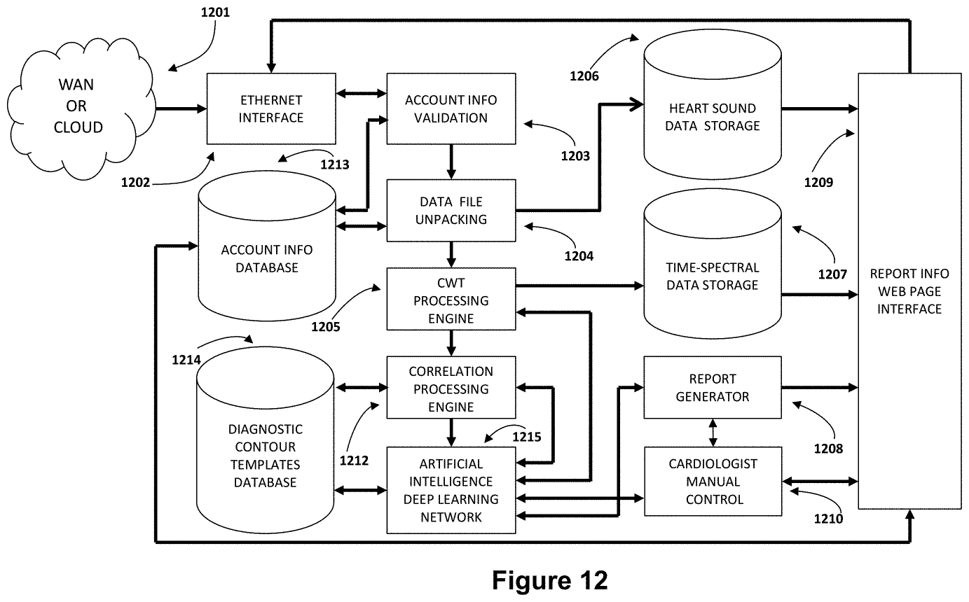

With reference to FIG. 12 the Cardiometric Processing Center performs the following task:
Ethernet Interface 1202 (multiple routing servers);
Account User Validation process using encrypted patient data 1203 (dual redundant secure accounting servers);
Unpacking of S1 thru S4 acoustic heart data 1204 (multi-port high speed servers),
Converting S1 thru S4 acoustic heart data to an image map using a continuous wavelet transform 1205 (server bank of supercomputers);
Multi-dimensional correlation between patient heart sound image contours and the CSI reference contours 1212 (server bank of supercomputers);
Deep Learning Neural Net determines which diagnostic template best matches the reference templates of abnormal or diseased heart function 1215 (server bank of supercomputers);
Database management of account information 1213, diagnostic contour templates 1214, heart sound data 1206, and time-frequency patient data 1207 (multiple banks of supercomputer database servers);
Webpage User interface server accessible by SmartPhone 1209 (multiple high speed web servers);
Report generator providing diagnostic data to webpage interface 1208 (supercomputer); and
A cardiologist manual control interface providing access to computational parameters and patient data 1210.
   The CSI processing center provides simultaneous processing of heart sound data from a plurality of remote sites, allowing delivery of DLNN access from any SmartPhone with online access. The hosted web sites 1209 allow viewing the patient input heart sound contour and the resulting match to a heart condition identified from comparison with the CSI contour database 1214. Additionally a CSR number is provided for easy indication of the severity of a diagnosed condition, thus alleviating the need for a health care professional to read the heart sound contours.

7. Cardiac Acoustic Training and Test Data Sets

The learning process associated with the CSI Deep Learning Neural Network is illustrated by the two layer example in FIG. 13. In one embodiment of this invention, a training data set consisting of acoustic heart data in the form of time series vectors (Cbad_1) for the 23 normal and abnormal heart conditions were created. Additionally, an independent acoustic heart test data set (Ctest_1) was created to validate the performance of the deep learning neural net. These data sets are Python and Matlab compatible.

The output of the example two layer neural net is given in Equation 5. The weights W and biases b are the only variables that effect the output ŷ 1301, 1302, 1303, 1304 and 1305. The loss function is given in Equation 6. Each iteration of the training process consists of the following steps:
Calculating the predicted output ŷ, known as Feedforward
Updating the weights and biases, known as Backpropagation
   Updates of the weights and biases are done by increasing or decreasing their values using a gradient descent method. Two thousand iterations are used to train the CSI neural net.

What is claimed is:

1. A system, method and apparatus comprising:
A mobile or clinical system that uses S1 thru S4 acoustic cardiac sounds to diagnose early, critical, and severe cardiac abnormalities or disease. The system uses a combination of wavelet time-frequency imaging, Deep Search Correlation, and Deep Learning Neural Networks to diagnose early heart conditions with 97% accuracy.
The system components and methods include:
a unique heart sound signature for a human individual;
a very low frequency acoustic sensor;
a sensor interface unit;
a sensor interface power source;
a USB compatible SmartPhone with WAN, LAN or WiFi connectivity;
a SmartPhone based CSI application software;
a remote super computer server farm;
a Wavelet based time-frequency analysis algorithm;
an AI Deep Learning Neural Network based cardiac diagnosis algorithm;
a custom neural network activity function named worm_1;
a Depth Search based Correlation algorithm;
a S1 thru S2 imaging contour templates collection for known cardiac conditions;
a normal, abnormal and diseased 500 member patient acoustic heart sound training data sets;
a cardiac severity rating algorithm and
a web page based report generator.

2. The system, apparatus and method of claim 1 wherein a plurality of sensors, interface units and SmartPhones are connected to a remote server farm over a LAN is used for heart condition diagnosis based on cardiac sound time-frequency contours.

3. The system, apparatus and method of claim 1 wherein a plurality of sensor, interface units and SmartPhones are connected to a remote server farm over a WAN is used for heart condition diagnosis based on cardiac sound time-frequency contours.

4. The system, apparatus and method of claim 1 wherein a plurality of sensors, interface units and SmartPhones are connected to a remote server farm over WiFi is used for heart condition diagnosis based on cardiac sound time-frequency contours.

5. The system, apparatus and method of claim 1 wherein a plurality of sensors, interface units and SmartPhones are connected to a remote server farm over the internet cloud is used for heart condition diagnosis based on cardiac sound time-frequency contours.

6. The system, apparatus and method of claim 1 wherein a CSI SmartPhone based application provides access to the Deep Learning Neural Net server farm.

7. The system, apparatus and method of claim 1 wherein a data set of 92 plus 3D synthesized cardiac sound image templates for known normal, abnormal, and disease heart conditions are used to form a correlation analysis library.

8. The system, apparatus and method of claim 1 wherein a test data set (Ctcor_1) of 500 3D synthesized cardiac sound image templates for known normal, abnormal, and disease heart conditions are used to test the correlation analysis library.

9. The system, apparatus and method of claim 1 wherein cardiac acoustic data for known normal and abnormal heart conditions form a training data set (Cbad_1) for the CSI deep learning neural net.

10. The system, apparatus and method of claim 1 wherein cardiac acoustic data for known normal and abnormal heart conditions form a test data set (Ctest_1) for the CSI deep learning neural net.

11. The system, apparatus and method of claim 1 wherein a cardiac severity rating calculation is used for early diagnosis and monitoring.

12. The system, apparatus and method of claim 1 wherein multiple neural network structures are used for early diagnosis and monitoring of cardiac condition.

13. The system, apparatus and method of claim 1 wherein the activity function Worm_1 is used in the Deep Learning Neural Net.

14. The system, apparatus and method of claim 1 wherein a remote CSI Processing Center is used to extend the deep learning neural net to remote SmartPhones.
